(19) United States
(12) Patent Application Publication
(10) Pub. No.: US 2007/0037979 A1
(43) Pub. Date: Feb. 15, 2007

(54) Preparation of rosuvastatin

(76) Inventors: Valerie Niddam-Hildesheim, Ein Vered (IL); Anna Balanov, Rehovot (IL); Natalia Shenkar, Petach Tiqva (IL); Shalom Shabat, Yavne (IL); Dalia Maidan-Hanoch, Kfar Yona (IL)

Correspondence Address: KENYON & KENYON LLP, ONE BROADWAY, NEW YORK, NY, 10004, US

(21) Appl. No.: 11/360,725

(22) Filed: Feb. 22, 2006

Related U.S. Application Data
(60) Provisional application No. 60/655,580, filed on Feb. 22, 2005.
(60) Provisional application No. 60/676,388, filed on Apr. 28, 2005.
(60) Provisional application No. 60/723,491, filed on Oct. 3, 2005.
(60) Provisional application No. 60/723,875, filed on Oct. 4, 2005.
(60) Provisional application No. 60/732,979, filed on Nov. 2, 2005.
(60) Provisional application No. 60/751,079, filed on Dec. 15, 2005.
(60) Provisional application No. 60/760,506, filed on Jan. 19, 2006.
(60) Provisional application No. 60/762,348, filed on Jan. 25, 2006.

(51) Int. Cl. A61K 31/505; C07C 69/66; C07D 239/42

(52) U.S. Cl. 544/330; 560/179

(57) ABSTRACT

Provided are processes for preparing intermediates of rosuvastatin and their use in preparation of rosuvastatin and rosuvastatin salts thereof.

### RELATED APPLICATIONS

This application claims the benefit of U.S. Provisional Application No. 60/655,580, filed Feb. 22, 2005; U.S. Provisional Application No. 60/676,388, filed Apr. 28, 2005; U.S. Provisional Application No. 60/723,491, filed Oct. 3, 2005; U.S. Provisional Application No. 60/723,875, filed Oct. 4, 2005; U.S. Provisional Application No. 60/732,979 filed Nov. 2, 2005; U.S. Provisional Application No. 60/751,079, filed Dec. 15, 2005; U.S. Provisional Application No. 60/760,506, filed Jan. 19, 2006; and U.S. Provisional Application No. Awaited, filed Jan. 25, 2006 (Attorney Docket No. 1662/71804).

### FIELD OF THE INVENTION

The invention is directed to processes for preparing intermediates of rosuvastatin and their use in preparation of rosuvastatin and rosuvastatin salts thereof.

### BACKGROUND

Complications of cardiovascular disease, such as myocardial infarction, stroke, and peripheral vascular disease account for half of all deaths in the United States. A high level of low density lipoprotein (LDL) in the bloodstream has been linked to the formation of coronary lesions which obstruct the flow of blood and promote thrombosis. [See Goodman and Gilman, The Pharmacological Basis of Therapeutics, 9^{th} ed., p. 879 (1996)]. Reducing plasma LDL levels has been shown to reduce the risk of clinical events in patients with cardiovascular disease and in patients who are free of cardiovascular disease but who have hypercholesterolemia. [Scandinavian Simvastatin Survival Study Group, 1994; Lipid Research Clinics Program, 1984a, 1984b.]

Statin drugs are currently the most therapeutically effective drugs available for reducing the level of LDL in the blood stream of a patient at risk for cardiovascular disease. This class of drugs includes, inter alia, compactin, lovastatin, simvastatin, pravastatin and fluvastatin.

The mechanism of action of statin drugs has been elucidated in some detail. The statin drugs disrupt the synthesis of cholesterol and other sterols in the liver by competitively inhibiting the 3-hydroxy-3-methyl-glutaryl-coenzyme A reductase enzyme (“HMG-CoA reductase”). HMG-CoA reductase catalyzes the conversion of HMG-CoA to mevalonate, which is the rate determining step in the biosynthesis of cholesterol. Consequently, HMG-CoA reductase inhibition leads to a reduction in the rate of formation of cholesterol in the liver. Decreased production of cholesterol causes an increase in the number of LDL receptors and corresponding reduction in the concentration of LDL particles in the bloodstream. Reduction in the LDL level in the bloodstream reduces the risk of coronary artery disease. [J.A.M.A. 1984, 251, 351-74].

Currently available statins include: lovastatin, simvastatin, pravastatin, fluvastatin, cerivastatin and atorvastatin, which are administered in their lactone form, as sodium salts or as calcium salts.

Rosuvastatin (7-[4-(4-fluorophenyl)-6-isopropyl-2-(N-methyl-N-methylsulfonylamino)pyrimidin-5-yl]-(3R,5S)-dihydroxy-(E)-6-heptenoic acid) calcium, an HMG-CoA reductase inhibitor can lower LDL-cholesterol and triglycerides levels more effectively than first generation statin drugs. Rosuvastatin calcium has the following chemical formula:

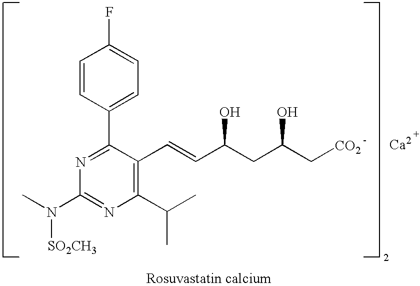

A number of relevant processes for preparation of rosuvastatin and salts thereof are disclosed. Rosuvastatin calcium, intermediates and their preparation are disclosed in U.S. Pat. No. 5,260,440, herein '440. WO 03/097614 discloses the synthesis of rosuvastatin from the late intermediate (3R)-3-(tert-butyldimethylsilyloxy)-5-oxo-6-triphenyl-phosphoralydene hexanate, an intermediate disclosed in '440. WO 03/087112 discloses the synthesis of rosuvastatin from a different intermediate, (3R)-3-(t-butyldimethylsilyloxy)-6-dimethoxyphosphinyl-5-oxohexanate. WO/0049014 discloses the synthesis of rosuvastatin using intermediates with other side chains via a Wittig reaction. EP 850,902 describes the removal of triphenylphosphine derivatives in mixtures.

Nevertheless, there remains a need in the art for processes of preparing rosuvastatin that are both cost effective and have fewer purification steps, thereby making them more suitable for industrial scale preparation.

### SUMMARY OF THE INVENTION

The present invention provides processes for the preparation of rosuvastatin and intermediates of rosuvastatin.

In one aspect of the present invention, a process is provided for preparing intermediate compound 17 of the following structure: 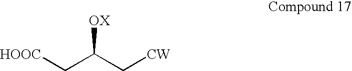
by partial hydrolysis of the diester, compound I, of the following structure: 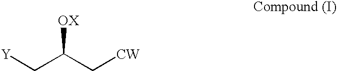
wherein Y is a C₁-C₄ ester, W is a carboxyl protecting group, and X is a hydroxyl protecting group. The process comprises: providing a solution of compound I and a polar solvent; combining the solution with a base to obtain a pH of about 10 to about 13; and recovering compound 17.

In another aspect of the present invention, a process for recovering compound 17 from the reaction mixture is provided. This process comprises: providing crude compound 17; partially evaporating the solvent; adding water; washing with a C₅-C₇ alkyl; extracting using an organic solvent selected from the group consisting of: saturated or aromatic C₅-C₁₂ hydrocarbons, mono-, di-, tri-(C₁ to C₄)alkyl substituted benzene; acidifying the mixture using an inorganic acid to a pH of about 7 to about 5; and recovering compound 17 from the organic phase.

Optionally, compound 17 may be recovered from the reaction mixture using techniques known to those skilled in the art.

In another aspect of the present invention, compound 17 prepared by the process of the present invention is used to prepare any downstream intermediate, rosuvastatin and pharmaceutically acceptable salts thereof by conventional means, for example as depicted in U.S. Pat. No. 5,260,440. For example, the following reaction scheme describes one method of converting compound 17 into rosuvastatin calcium, wherein compounds 17 to 22 are represented by number:

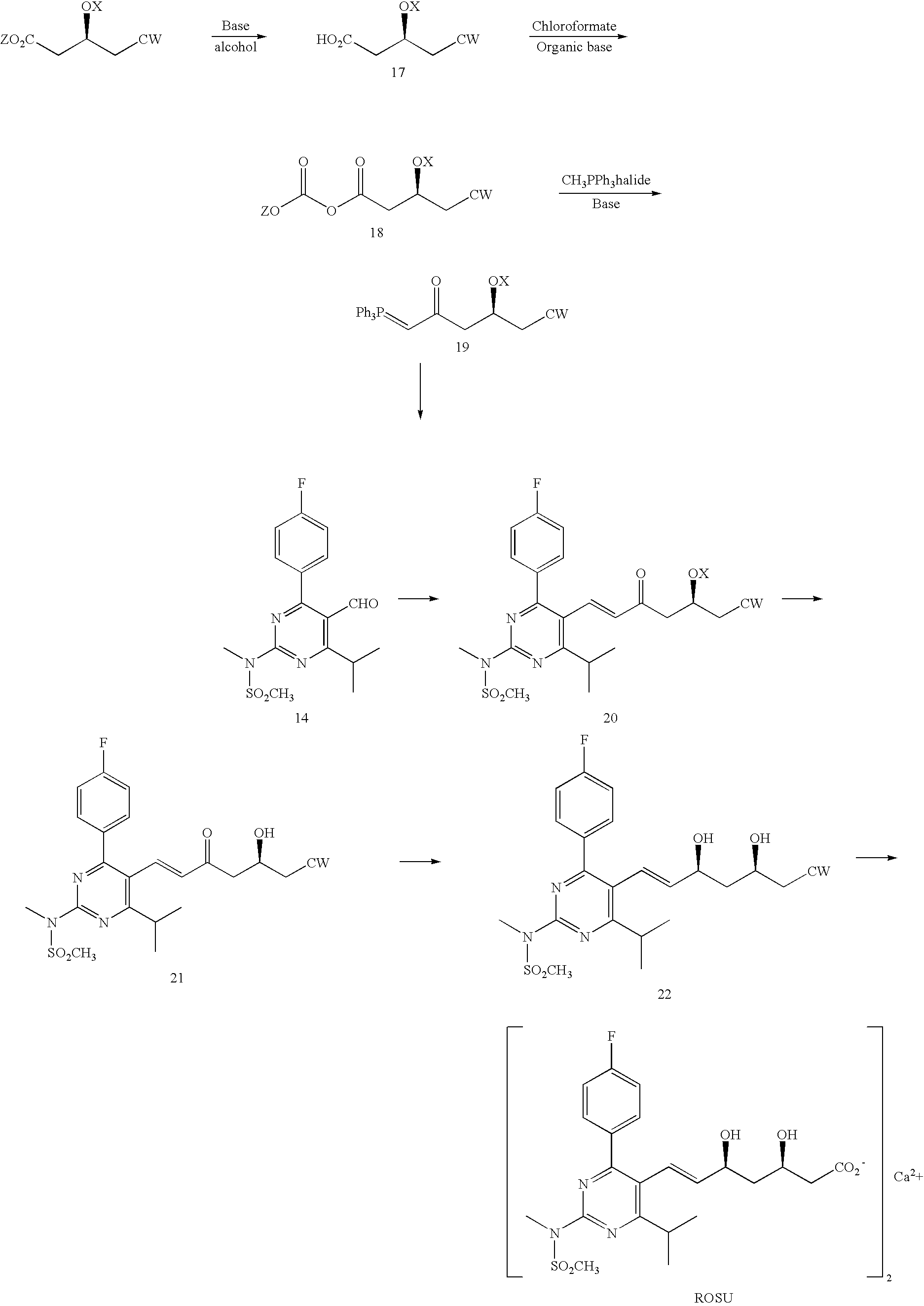

### Preparation of Rosuvastatin through Intermediates

wherein W represents a carboxyl protecting group, Z is a C₁-C₆ or C₈ alkyl group and is an hydroxyl protecting group.

In another aspect, the present invention provides a process of preparing intermediate compound 18, as shown in the following structure: 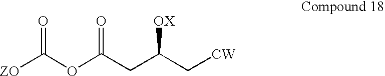
wherein W is a carboxyl protecting group, and X is a hydroxyl protecting group, and Z is a C₁-C₈ alkyl. The process comprises: adding a first solution, comprising compound 17, a first organic solvent and a base, to a second solution comprising a mono-, di-, tri-(C₁ to C₄)alkyl substituted benzene chloroformate, saturated or aromatic C₅-C₁₂ chloroformate or C₁₋₈ alkyl chloroformate and a second organic solvent to obtain a reaction mixture while maintaining a temperature of about −50° C. to about −10° C.; and maintaining the reaction mixture for a sufficient period of time to obtain compound 18.

Optionally, compound 18 may be recovered from the reaction mixture using techniques known to those skilled in the art.

In another aspect of the present invention, compound 18 prepared by the process of the present invention is used to prepare any downstream intermediate, rosuvastatin and pharmaceutically acceptable salts thereof.

Compound 18 may be converted into compound 19, of the following structure: 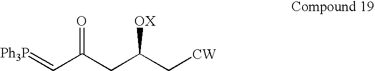
wherein X is any hydroxyl protecting group and W is any carboxyl protecting group, by methods known in the art, for example by gradually adding a solution of compound 18 in toluene to a cooled solution comprising: methyl triphenylphosphonium bromide, THF, and a butyllithium while maintaining the temperature at about −60° C. to obtain a reaction mixture; and maintaining the reaction mixture at a maximum temperature of about −20° C. for a sufficient amount of time to obtain compound 19. [See U.S. Pat. No. 5,260,440]

In another aspect of the present invention, a process is presented for the preparation of compound 20 through the Wittig condensation of compound 19 and compound 14, as shown below: 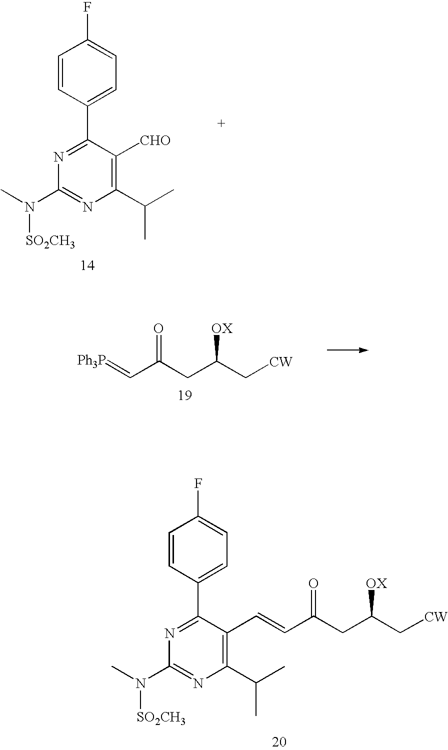
wherein W is a carboxyl protecting group and X is a hydroxyl protecting group. This process comprises: providing compound 19, compound 14 and a suitable organic solvent other than acetonitrile, to obtain a reaction mixture in an inert atmosphere such as argon or nitrogen; and heating the reaction mixture at about 70° C. to about reflux for a sufficient period to obtain compound 20.

Optionally, compound 20 may be recovered from the reaction mixture using techniques known to those skilled in the art.

In another aspect of the present invention, compound 20 prepared by the process of the present invention is used to prepare any downstream intermediate, rosuvastatin and pharmaceutically acceptable salts thereof.

In another aspect of the present invention, a process for recovering compound 21 is provided. This process comprises: providing a two-phased system comprising a mixture of a non-polar aliphatic solvent and a non-polar aromatic solvent and a mixture of a lower aliphatic alcohol and water, each in an amount of about 4 to about 6 volumes relative to compound 21 and crude compound 21; washing the non-polar phase with a mixture of lower aliphatic alcohol and water; and recovering compound 21 from the organic phase.

Subsequent reduction of intermediate compound 21 to form compound 22 is performed under conditions known to those skilled in the art. Rosuvastatin may be obtained upon saponification of compound 22. In addition, the rosuvastatin prepared by the process of the invention may be converted to a pharmaceutically acceptable salt, such as a calcium salt.

In another aspect, the present invention provides a process for preparing rosuvastatin, and pharmaceutically acceptable salts thereof, by converting compound 17 into rosuvastatin. This process comprises:
a. providing a solution of compound I and a polar solvent;
b. combining the solution with a base to obtain a pH of about 10 to about 13 to form a first solution comprising compound 17;
c. adding a second solution comprising a mono-, di-, tri-(C₁ to C₄)alkyl substituted benzene chloroformate, saturated or aromatic C₅-C₁₂ chloroformate or C₁-C₈ alkyl chloroformate and an organic solvent to obtain a reaction mixture while maintaining a temperature of about −50° C. to about −10° C.;
d. maintaining the reaction mixture for a sufficient period of time to obtain compound 18;
e. converting compound 18 into compound 19;
f. providing compound 19, compound 14 and a suitable organic solvent other than acetonitrile, to obtain a reaction mixture in an inert atmosphere such as argon or nitrogen;
g. heating the reaction mixture at about 70° C. to about reflux for period to obtain compound 20;
h. converting compound 20 into compound 21;
i. optionally recovering compound 21 by providing a two-phased system comprised of a mixture of a non-polar aliphatic solvent and a non-polar aromatic solvent and a mixture of a mixture of a lower aliphatic alcohol and water, each in an amount of about 4 to about 6 volumes relative to compound 21 and crude compound 21, washing the non-polar phase with a mixture of lower aliphatic alcohol and water, and recovering compound 21 from the organic phase;
j. converting compound 21 into compound 22; and
k. converting compound 22 into rosuvastatin.
   Optionally, compound 17 may be recovered from step b. by partially evaporating the solvent from the first solution, adding water, washing with a C₅-C₇ alkyl, extracting using an organic solvent selected from the group consisting of: saturated or aromatic C₅-C₁₂ hydrocarbons, mono-, di-, tri-(C₁ to C₄)alkyl substituted benzene, acidifying the mixture using an inorganic acid to a pH of about 7 to about 5; and recovering compound 17 from the organic phase. The recovered compound 17 may then be combined with a first organic solvent and a base to form the first solution comprising compound 17.

Rosuvastatin obtained by the processes of the invention may be converted to a pharmaceutically acceptable salt of rosuvastatin, preferably the calcium salt.

### DETAILED DESCRIPTION OF THE INVENTION

As used herein KF refers to Karl Fisher titration, a widely used analytical method for quantifying water content.

As used herein, RT refers to room temperature and includes temperatures of about 25±5° C.

The carboxyl protecting group in the structures within the present application may be any suitable carboxyl protecting group, such as esters, amides, benzenes or hydrazides. More preferably, the carboxyl protecting group is an ester, and most preferably is a tert-butyl ester in the structures of the present inventions. Some typical examples of a hydroxyl protecting group include methoxymethyl esters, tetrahydropyranyl ether, trimethylsilyl ether, tertbutyl diphenyl silyl, Stannum derivatives, and acetate ester. Preferably the tri(C₁-C₆ alkyl)silyl is tri(C₁ to C₄ alkyl)silyl, even more preferably trimethylsilyl, or tert-butyldimethylsilyl (TBDMS), with TBDMS being especially preferred. More carboxyl or hydroxyl protecting groups are described in “Protective Groups in Organic Synthesis” by T. W. Greene, John Wiley & Sons, Inc. (1981).

As used herein, lower aliphatic alcohols include C₁ to C₄ alcohols.

When used herein, the suffix “TB” describes intermediate compounds described in the summary, wherein R is t-butyl. For example, the term “17TB” refers to intermediate compound 17 wherein R is t-butyl. The suffix “M” describes intermediate compounds wherein R is methyl. For example, the term “17M” refers to intermediate compound 17, wherein R is methyl. The suffix “TBPH” describes compounds herein wherein R is t-butyl and PH is phenyl. The suffix “TBRE” describes compounds herein wherein R is tert-butyl and RE is rosuvastatin ester. The suffix “TBDMS” describes compounds herein wherein R is t-butyl and DMS is tert-butyl dimethyl silyl.

The invention provides improved processes for the preparation of rosuvastatin and intermediates thereof in high yield using cost effective reagents. The processes of the invention provide for the quantitative conversion of reagents and decreased formation of by-products, resulting in a process for preparing rosuvastatin requiring fewer purification steps. Examples in specific cases are dispersed throughout.

In one aspect of the present invention, a process is provided for preparing intermediate compound 17, of the following structure: 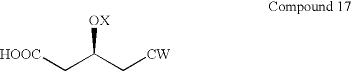
by partial hydrolysis of the diester, compound I, of the following structure: 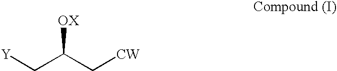
wherein Y is a C₁-C₄ ester, W is a carboxyl protecting group, and X is a hydroxyl protecting group. The process comprises: providing a solution of compound I and a polar solvent; combining the solution with a base to obtain a pH of about 10 to about 13; and recovering compound 17. In this process, the synthesis of compound 17 enables the production of a monoacid derivative with little contamination of the diacid derivative.

Polar solvents can be selected from the group consisting of: C₁₋₄ alcohols, nitrites, acetone, dioxane, and THF, most preferably, methanol and ethanol. Polar solvent is in amount of about 2 to about 15 volumes, preferably about 5 to about 10, and most preferably 5 volumes relative to compound I.

The base used is any suitable base, which can be selected from the group consisting of: mono-, di-, tri-(C₁₋₄ alkyl)amino pyridines, mono-, di-, tri-(C₁₋₄ alkyl)amines, alkali metals, alkali earth hydroxides, alkali earth alkooxides, and C₁₋₄ alkyl lithium carbonates. Preferably, the base is at least one of sodium hydroxide, potassium hydroxide, or lithium hydroxide, most preferably sodium hydroxide.

Preferably, the base is in a concentration of about 0.9 to about 1.8 volumes, most preferably about 1.2 volumes relative to compound I. In a particularly preferred embodiment, the base is added drop-wise to a solution of Compound (I). The base may be added in portions to maintain the pH at this level. The amount of base required to effect the reaction will depend on the scale of the reaction, and may easily be determined by one skilled in the art with little or no experimentation using such techniques as TLC.

Preferably, the reaction mixture is heated at a temperature of about 30° C. to about 70° C. Most preferably, the reaction mixture is heated at about 45° C. to about 55° C. Heating is for a period of time, will depend on scale and mixing procedures, and can be determined by one skilled in the art by measuring the absence of the limiting reagent using such techniques such as HPLC or TLC. For example, when about 288 mmol of compound I is used, the heating time is about 1 hour to about 10 hours, and preferably about 7 hours.

In another aspect of the present invention, a process for recovery of compound 17 from the reaction mixture is provided. This process comprises: providing crude compound 17; partially evaporating the solvent; adding water; washing with a C₅₋₇ alkyl; extracting using an organic solvent selected from the group consisting of: saturated or aromatic C₅-C₁₂ hydrocarbons, mono-, di-, tri-(C₁ to C₄)alkyl substituted benzene; acidifying the mixture using an inorganic acid to a pH of about 7 to about 5; and recovering compound 17 from the organic phase.

The water used is preferably in an amount of about 2 to about 10 volumes, most preferably 4 volumes relative to the crude compound 17. Preferably, the C₅₋₇ alkyl is hexane. The washing may be in portions, preferably about 2. The organic solvent is preferably toluene. Any inorganic acid may be used for acidification, preferably HCl. Preferably, acidifying is to a pH of about 6. Recovery from the organic phase may be by drying, such as over MgSO₄.

In another aspect of the present invention, compound 17 prepared by the process of the present invention is used to prepare any downstream intermediate, rosuvastatin and pharmaceutically acceptable salts thereof by conventional means, for example as depicted in U.S. Pat. No. 5,260,440. For example, the following reaction scheme describes one method of converting compound 17 into rosuvastatin calcium, wherein compounds 17 to 22 are represented by number:

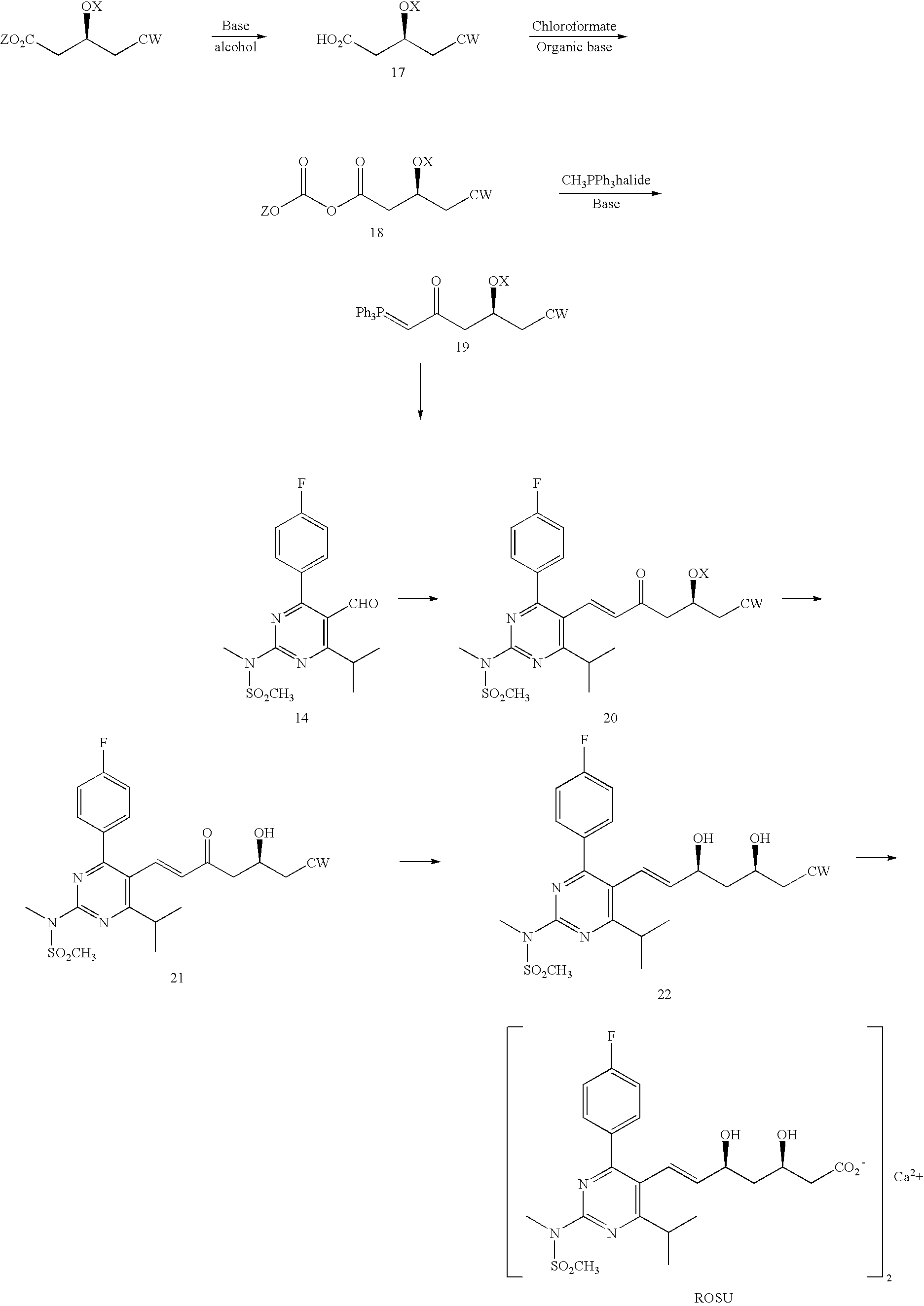

### Preparation of Rosuvastatin through Intermediates

wherein W represents a carboxyl protecting group, Y is a C₁-C₆ or C₈ alkyl group and X is an hydroxyl protecting group.

In another aspect of the present invention, a process is provided for preparing intermediate compound 18, as shown in the following structure: 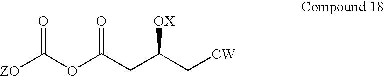
wherein W is a carboxyl protecting group, and X is a hydroxyl protecting group, and Z is a C₁₋₈ alkyl. The process comprises: adding of a first solution comprising compound 17, a first organic solvent and a base, to a second solution comprising a mono-, di-, tri-(C₁ to C₄)alkyl substituted benzene chloroformate, saturated or aromatic C₅-C₁₂ chloroformate or C₁₋₈ alkyl chloroformate and a second organic solvent to obtain a reaction mixture while maintaining a temperature of about −50° C. to about −10° C.; and maintaining the reaction mixture for a sufficient period of time to obtain compound 18.

The base may be any suitable organic base, including, but not limited to, di-(C₁ to C₄ alkyl) pyridine, wherein the alkyl group may be the same or different, mono-15, di-, or tri-(C₁ to C₄ alkyl)amines, wherein the alkyl groups can be the same or different, alkaline earth metals, alkaline earth hydroxides, alkaline earth alkooxides, C₁₋₄ alkyl lithium. Preferably, the base is a C₁-C₄ trialkylamine, and most preferably is triethylamine.

The first and second organic solvents suitable for use in the process of the invention include, but are not limited to, saturated or aromatic C₅₋₁₂ hydrocarbons, mono-, di-, tri-,(C₁₋₄)alkyl substituted benzenes, and benzenes. For example, THF, toluene, methylene chloride, diethylether, benzene, and chloroform may be used. Toluene and THF are preferred organic solvents. The same organic solvent is preferably used for both the first and second organic solvent.

Preferably the mono-, di-, tri-(C₁ to C₄)alkyl substituted benzene chloroformate, saturated or aromatic C₅-C₁₂ chloroformate or C₁₋₈ alkyl chloroformate is a C₁₋₄ alkyl chloroformate, more preferably ethyl chloroformate or methyl chloroformate, with ethyl chloroformate being particularly preferred. The molar ratio of the chloroformate to compound 17 in the reaction mixture is about 1 mole to about 3 moles, and is preferably about 1 mol to about 1.5 mol.

The first solution is combined with the second solution at a temperature of about −50° C. to about −10° C., more preferably at a temperature of −50 to about −30° C. and most preferably at a temperature of about −45° C. to about −40° C. Preferably the solutions are combined over a period of about 30 minutes. The reaction mixture is maintained by gradual heating to about −10° C. to about 30° C., and more preferably to about 0° C. The sufficient period of time required to obtain compound 18 will depend on, for example, scale and mixing procedures. This can be determined by one skilled in the art by measuring the absence of the limiting reagent using such techniques such as HPLC or TLC, preferably TLC. Optionally, the reaction mixture can then be quenched, preferably with water.

Optionally, compound 18 may be recovered from the reaction mixture using techniques known to those skilled in the art. Preferably, compound 18 is recovered by separating the organic layer formed during quenching from the reaction mixture and washing the organic layer with a mild base (pH 7-11), such as NaHCO₃ The reaction mixture may be washed by adding NaCl. The organic layer is then dried, for example with a metal salt, preferably Na₂SO₄ or MgSO₄. The solvent is then evaporated to obtain compound 18. Alternatively, the reaction mixture is filtered to remove the salts formed during the reaction.

Preparing compound 18 according to the process of the invention reduces the formation of a symmetric anhydride impurity and allows a quantitative formation of a mixed anhydride product. In addition, the process of this invention can be used easily on an industrial scale as extreme temperatures are not used, in contradistinction to U.S. Pat. No. 5,260,440 where −70° C. to −85° C. are ideally used

In another aspect of the present invention, compound 18 prepared by the process of the present invention is used to prepare any downstream intermediate of rosuvastatin or pharmaceutically acceptable salts thereof.

Compound 18 may be converted into compound 19, of the following structure: 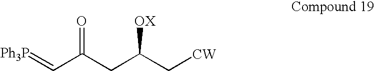
wherein X is any hydroxyl protecting group and W is any carboxyl protecting group, by methods known in the art, for example by gradually adding a solution of compound 18 in toluene to a cooled solution comprising: methyl triphenylphosphonium bromide, THF, and a butyllithium while maintaining the temperature at about −60° C. to obtain a reaction mixture; and maintaining the reaction mixture at a maximum temperature of about −20° C. for a sufficient amount of time to obtain compound 19. [See U.S. Pat. No. 5,260,440]

In another aspect of the present invention, compound 19 prepared by the process of the present invention can be used to prepare any downstream intermediate, rosuvastatin and pharmaceutically acceptable salts thereof.

In another aspect of the present invention, a process is presented for the preparation of compound 20 through the Wittig condensation of compound 19 and compound 14, as shown below: 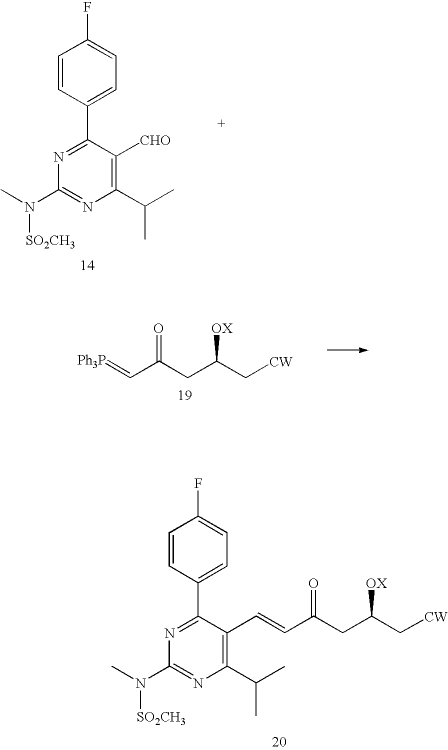
wherein W is a carboxyl protecting group and X is a hydroxyl protecting group. This process comprises: providing compound 19, compound 14 and a suitable organic solvent other than acetonitrile, to obtain a reaction mixture in an inert atmosphere such as argon or nitrogen; and heating the reaction mixture at about 70° C. to about reflux for period to obtain compound 20.

The organic solvent can be any suitable organic solvent including, but not limited to, saturated or aromatic C₅-C₁₂ hydrocarbons, mono-, di-, tri-(C₁ to C₄ alkyl substituted benzenes, and benzenes. Preferably, the organic solvent is toluene.

Compound 19 is in an amount of 1.5 equivalents relative to compound 14, while the organic solvent other than acetonitrile is about 10 volumes relative to compound 14. Heating the reaction mixture is preferably to about 70° C. to about 110° C., most preferably about 100° C. The period of time necessary depends on the scale and temperature of the process and may be determined easily by anyone skilled in the art.

Upon obtaining compound 20, an assay may be performed to establish the amount of compound 20 in the crude compound 20 produced by the process of this invention. Typically, about 50% compound per weight is obtained as detected by HPLC by comparing to a standard. This assay measures contamination of compound 20 by salts or non-UV impurities, or formation of by-products of degradation, especially in the case of the Wittig reaction. Regardless of these impurities, compound 20 formed from this process may be used directly without further purification in the next step to form compound 21.

Overall, this process results in a quantitative conversion of starting materials. Preferably, compound 14 is present in a quantity of less than 5% as measured by HPLC, and most preferably less than 2% as measured by HPLC.

Triphenylphosphine oxide is formed as a by-product of the reaction, and can be removed from the reaction mixture. Preferably, triphenylphosphine oxide is removed by forming a complex with a metal salt by combining a metal salt, preferably anhydrous magnesium chloride with the reaction mixture, as disclosed in EP Patent No. 0850902A1, and isolating compound 20 by heating to about 100° C., cooling to about 0° C., filtering, washing with water or toluene and evaporating the solvent.

In another aspect of the present invention, compound 20 prepared by the process of the present invention is used to prepare any downstream intermediate of rosuvastatin and pharmaceutically acceptable salts thereof.

Compound 21 may be prepared by the deprotection of the hydroxyl group of compound 20, as disclosed in WO 2003/097614 A2 as shown below: 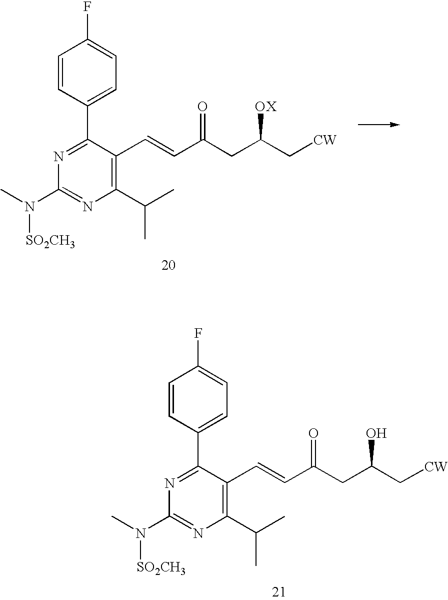
wherein W is a carboxyl protecting group and X is a hydroxyl protecting group. In one example, a solution of compound 20 in methanol, THF or acetonitrile is combined with a deprotecting agent, such as a fluoride ion source or an inorganic acid aside from HF, to obtain a reaction mixture; and the reaction mixture is maintained for a sufficient time and temperature to obtain compound 21.

In another aspect of the present invention, a process for recovery of compound 21 is provided. This process comprises:
a. providing a two-phased system comprised of a mixture of a non-polar aliphatic solvent and a non-polar aromatic solvent and a mixture of a mixture of a lower aliphatic alcohol and water, each in an amount of about 4 to about 6 volumes relative to compound 21 aid crude compound 21;
b. washing the non-polar phase with a mixture of lower aliphatic alcohol and water; and
c. recovering compound 21 from the organic phase.

Compound 21, having a purity of greater than about 80%, preferably about 90% (as determined by HPLC) and a yield of greater than about 90%, preferably greater than about 95%, may be obtained using this recovery method.

Preferably, the non-polar aliphatic solvent, non-polar aromatic solvent, lower aliphatic alcohol and water in step a. are each in an equal volume of about 5 volumes relative to compound 21. Preferably, the non-polar aliphatic solvent is heptane. Preferably, the non-polar aromatic solvent is toluene. Preferably, the lower aliphatic alcohol is ethanol. Preferably, providing the two-phase system of step a. includes mixing the reagents of step a. at room temperature until a clear solvent is obtained and allowing the mixture to separate into phases.

Washing the non-polar phase with the mixture of polar solvent and water is preferably in stages, where 5 times should be sufficient. In a more preferred embodiment, 4 portions of ethanol and water is used. Preferably, the ratio of ethanol to water is in a ratio of about 2:1 by volume. Preferably, the ethanol is in an amount of about 4 to about 6 volumes, preferably 5 volumes relative to compound 21 while the water is in an amount of about 8 to about 12 volumes relative to compound 21, preferably about 10 volumes. Preferably, fractions 2 through 5 from 5 fractions are collected, combined and concentrated, preferably under reduced pressure, to obtain an oily residue of compound 21.

The recovery process of compound 21 described above allows for the crystallization of compound 22 after stereoselective reduction of compound 21. The production of compound 22 in solid form resulting from the purification of compound 21 allows rosuvastatin to be further purified, if desired. Crystallization of compound 21 may further reduce the impurities present; however, such crystallization may not provide a satisfactory yield.

Subsequent reduction of intermediate compound 21 to form compound 22, shown in the following: 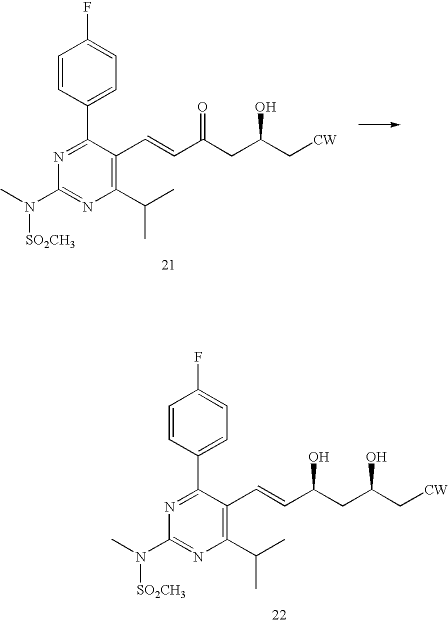
wherein W is a carboxyl protecting group and X is a hydroxyl protecting group. This process is performed under conditions known to those skilled in the art, and is preferably performed using diethylmethoxyborane in THF and sodium borohydride.

Rosuvastatin may be obtained upon saponification of compound 22.

In another aspect, the present invention provides a process for preparing rosuvastatin, and pharmaceutically acceptable salts thereof, by converting compound 17 into rosuvastatin. This process comprises:
a. providing a solution of compound I and a polar solvent;
b. combining the solution with a base to obtain a pH of about 10 to about 13 to form a first solution comprising compound 17;
c. adding a second solution comprising a mono-, di-, tri-(C₁ to C₄)alkyl substituted benzene chloroformate, saturated or aromatic C₅-C₁₂ chloroformate or C₁₋₈ alkyl chloroformate and an organic solvent to obtain a reaction mixture while maintaining a temperature of about −50° C. to about −10° C.;

d. maintaining the reaction mixture for a sufficient period of time to obtain compound 18;
e. converting compound 18 into compound 19;
f. providing compound 19, compound 14 and a suitable organic solvent other than acetonitrile, to obtain a reaction mixture in an inert atmosphere such as argon or nitrogen;

g. heating the reaction mixture at about 70° C. to about reflux for period to obtain compound 20;
h. converting compound 20 into compound 21;
i. optionally recovering compound 21 by providing a two-phased system comprised of a mixture of a non-polar aliphatic solvent and a non-polar aromatic solvent and a mixture of a mixture of a lower aliphatic alcohol and water, each in an amount of about 4 to about 6 volumes relative to compound 21 and crude compound 21, washing the non-polar phase with a mixture of lower aliphatic alcohol and water, and recovering compound 21 from the organic phase;
j. converting compound 21 into compound 22; and
k. converting compound 22 into rosuvastatin.

Optionally, compound 17 may be recovered from step b. by partially evaporating the solvent from the first solution, adding water, washing with a C₅₋₇ alkyl, extracting using an organic solvent selected from the group consisting of: saturated or aromatic C₅-C₁₂ hydrocarbons, mono-, di-, tri-(C₁ to C₄)alkyl substituted benzene, acidifying the mixture using an inorganic acid to a pH of about 7 to about 5; and recovering compound 17 from the organic phase. The recovered compound 17 may then be combined with a first organic solvent and a base to form the first solution comprising compound 17.

Rosuvastatin obtained by the processes of the invention may be converted to a pharmaceutically acceptable salt of rosuvastatin, preferably the calcium salt. [See e.g. U.S. Pat. No. 5,260,440]. The process of converting rosuvastatin into its pharmaceutically acceptable salt includes contacting rosuvastatin with calcium hydroxide, or with a stronger base such as sodium hydroxide. The base is preferably combined dropwise with a reaction mixture of rosuvastatin at a suitable temperature, such as a temperature of about 25° C.±5° C. The reaction mixture may be washed with a suitable water immiscible organic solvent. Suitable water immiscible organic solvents include, but are not limited to, hydrocarbons; preferably the water immiscible organic solvent is toluene. The water immiscible organic solvent may be removed by phase separation. Remaining water immiscible organic solvent may be removed by distillation of the reaction mixture, preferably at a temperature of about 40° C. to about 45° C. under reduced pressure (below about 50 mmHg).

The reaction mixture may then be combined with an alkali metal, including a source of calcium such as calcium chloride or calcium acetate, to form the salt of rosuvastatin. [See e.g. U.S. Pat. No. 6,777,552]. For example, calcium chloride may be added dropwise to a reaction mixture of rosuvastatin at a suitable temperature, such as a temperature of about 35° C. to about 45° C., and preferably at about 40° C., over a period of about thirty to about ninety minutes. Active carbon may be combined with a reaction mixture of rosuvastatin to remove impurities from the reaction mixture. If active carbon is used during the conversion of rosuvastatin into its pharmaceutically acceptable salt, the active carbon may be used before or after contacting rosuvastatin with an alkali metal.

The conversion of rosuvastatin into its pharmaceutically acceptable salt may also include filtering the reaction mixture. The reaction mixture may be filtered, such as with Synter and Hyflo®, before or after washing with a water immiscible organic solvent.

The present invention, in certain of its embodiments, is illustrated by the following non-limiting examples.

All purities mentioned herein refer to a yield per weight quantification, measured by comparing HPLC of the product versus known standard.

### EXAMPLES

### Example 1

### Preparation of Compound 17TB

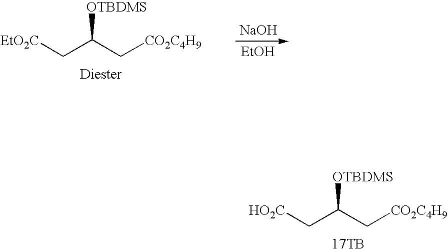

A 1 liter flask, equipped with a condenser, a mechanical stirrer, a pH-meter and a thermometer, was charged with t-butylethyl glutaric acid TBDMS protected (100 g, 288 mmol) and absolute EtOH (500 ml), forming a reaction mixture. The reaction mixture was heated to 50° C., and NaOH 1N (115.2 ml) was added dropwise. The pH measured 12.8.

After 1 hour at this temperature, the pH measured 10.59. Additional NaOH 1N (115.2 ml) was added. The pH measured 12.25. After 1 hour, additional NaOH 1 N (115.2 ml) was added.

The reaction mixture was maintained at 50° C. for 7 hours, until the starting material was not detected by TLC. The reaction mixture was cooled to room temperature, and evaporated to a final volume of 300 ml. H₂O (400 ml) and EtOH (95%, 50 ml) were added to the reaction mixture. The reaction mixture was washed twice with hexane (300 ml each).

Toluene was added (300 ml) to the aqueous phase, and the reaction mixture was neutralized with HCl (32%) to a pH of 6. Two additional extractions with toluene were performed (300 ml each). The toluene layers were combined, dried with MgSO₄ (approx 12 g), and evaporated, yielding 78.3 g (85% yield) of a yellow oil.

### Example 2

### Preparation of Compound 18TB

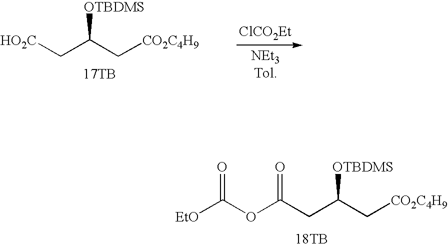

A 2 L flask was charged with a first solution of ethyl chloroformate (16.44 ml) in 900 ml of dry toluene (KF=less than 0.01%) and the solution was cooled to −45° C. A reaction mixture was formed by adding dropwise through a dropping funnel a second solution of compound 17TB (50 g) and Et₃N (26.06 ml) in 100 ml of toluene dropwise through a dropping funnel to the first solution over a period of about 30 minutes, so that the temperature of the reaction mixture was maintained at −45 to −40° C.

The reaction mixture was slowly heated to 0° C. over a period of 1.5 hours and then quenched with water. The reaction mixture was immediately transferred to a 2 L separation funnel, and the organic layer was washed with NaHCO₃ (saturated, 250 ml) and NaCl (saturated, 250 ml), and dried with MgSO₄. The solvent was evaporated and the residue was used for the next stage without any purification.

### Example 3

### Preparation of Compound 19TBPH

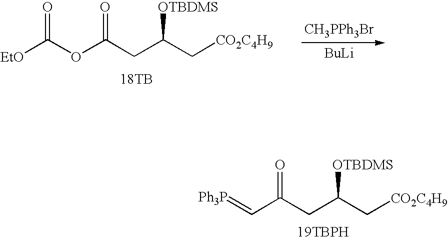

Methyl triphenyl phosphonium bromide (224.3 g) was suspended in THF(600 ml), and BuLi (1.6 M, 392.5 ml) was added over a period of 30 minutes at a temperature of about −55 to −50° C. The reaction mixture was then heated to about 0° C. over a period of 1.5 hours, and then cooled to about −60° C.

A solution of anhydride compound 18TB (122.6 g, 314 mmol) in toluene (360 ml) was added dropwise to the reaction mixture over a period of about two hours, while the temperature of the reaction mixture was maintained at about −55 to −65° C. The reaction mixture was heated to about 0° C. over a period of 1.5 hours, and quenched with water (250 ml). The aqueous phase was separated, and the product was extracted from the aqueous phase using toluene (100 ml). Both organic layers were mixed together and washed with NaHCO₃ (saturated, 2×100 ml) and NaCl (2×100 ml). The organic phase was kept overnight on Na₂SO₄ at about −25° C. and the solvent evaporated before use.

### Example 4

### Preparation of Compound 20TB by Wittig Reaction from 19TBPH

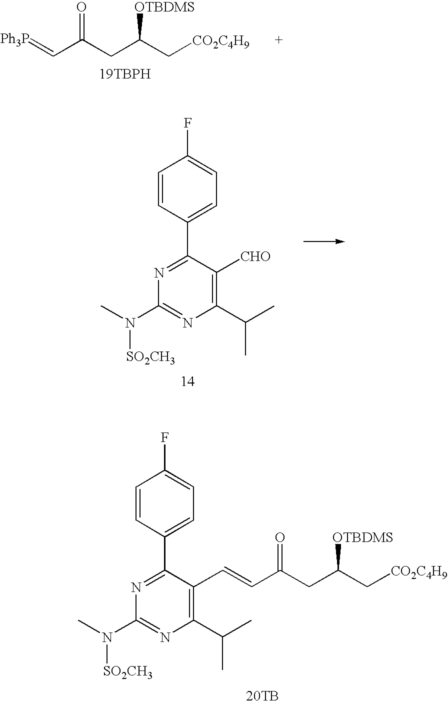

A 100 ml flask, protected from light and provided with N₂ flow was charged with compound 14 (3.6 g, 10.5 mmol), compound 19TBPH (9.05 g, 15.7 mmol), and dry toluene (36 ml, 10 vol relative to compound 14). The reaction mixture was heated to about 100° C. for 19.5 hrs. A sample of the reaction mixture was analyzed by HPLC, and contained 1.7% of compound 14.

Anhydrous MgCl₂ (2 g, 2 equivalents relative to compound 19TBPH) was added to the reaction mixture and the reaction mixture was stirred at 100° C. for 2 hrs. The reaction mixture was cooled to 0° C. for 2 hours, and filtered without washing the solid. A filtrate was obtained and was washed twice with H₂O (100 ml each) and the solvent was evaporated, yielding 7.56 g of a brown solid.

### Example 5

### Preparation of Compound 20M by Wittig Reaction from 19M

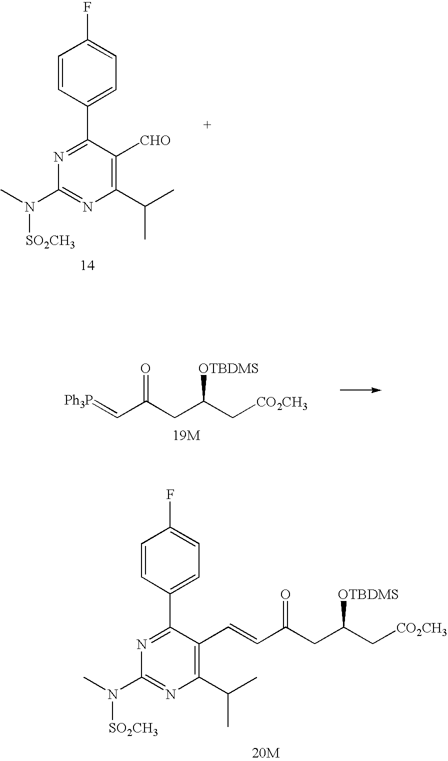

A 250 ml flask, protected from light and provided with N₂ flow was charged with compound-14 (4.38 g, 12.5 mmol), compound 19M (10 g, 18.7 mmol), and extra dry toluene (100 ml). The reaction mixture was heated to about 100° C. for 15 hrs. After the completion of the reaction, anhydrous MgCl₂ (4.8 g, 2.7 eq.) was added to the reaction mixture and the reaction mixture was heated for 2 hours at about 100° C. The reaction mixture was cooled to 0° C. over a period of about 2 hours, filtered, and washed with 45 ml of toluene, yielding 12.73 g of a viscous oil.

### Example 6

### Preparation of Compound 21TB in HCl/Methanol

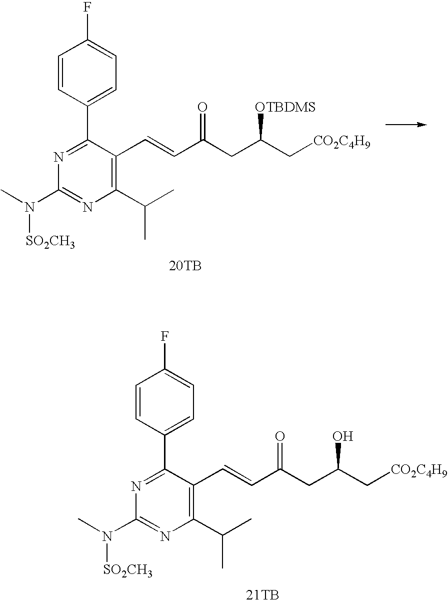

A mixture of HCl (32% in water, 1 mL), water (0.5 mL) and methanol (8 mL) was added dropwise to a solution of compound 20TB (2 g) in methanol (10 mL). The reaction mixture was stirred at 30° C. for about 1.5 hours, until TLC (Hexane/EtAc, 4:1) indicated full consumption of the starting material.

Ethyl acetate (150 mL) was added to the reaction mixture and the reaction mixture was washed with a saturated NaHCO₃ solution (50 mL×2), forming an organic layer. The organic layer was dried over MgSO₄ and the solvent was removed under reduced pressure, yielding compound 21TB (1.72 g).

### Example 7

### Preparation of Compound 21TB in HCl/THF

A mixture of HCl (32% in water, 0.57 g), water (2 mL), and THF (17.5 mL) was prepared. 5.4 mL of this mixture were added dropwise to a solution of compound 20TB (2.7 g) in THF (8.1 mL). The reaction mixture was stirred at ambient temperature overnight, until monitoring of the reaction by TLC indicated completion of the reaction.

Ethyl acetate (20 mL) was added to the reaction mixture and the reaction mixture was washed with water (20 mL). An aqueous layer formed, and was extracted with ethyl acetate (20 mL). The organic layers were combined and washed with an aqueous solution of Et₃N (2×5 mL) at a pH of about 10.5. The organic layer was dried over MgSO₄ and the solvent was removed under reduced pressure, yielding an oil of compound 21TB (2.03 g).

### Example 8

### Preparation of Compound 21TB with Tetrabutylammonium Fluoride/THF

Compound 20TB (5 g) was dissolved in THF (40 mL). Tetrabutylammonium fluoride in THF (8.46 ml, 1 M solution) was added dropwise to the solution, forming a reaction mixture. The reaction mixture was stirred for about 1 hour at room temperature. The solvent was removed under reduced pressure. Toluene (300 ml) was added to the solution. The solution was washed three times with a NaHCO₃ saturated solution (50 mL) and concentrated under reduced pressure, yielding compound 21TB.

### Example 9

### Preparation of Compound 21TB by TBDMS Deprotection with CsF, K₂CO₃ and NH₂OH.HCl

Compound 20TB (0.3 g) was dissolved in acetonitrile (10 ml) at room temperature. CsF (70 mg), K₂CO₃ (300 mg), and NH₂OH.HCl (160 mg) were added to the solution, forming a reaction mixture. The reaction mixture was heated at about 75° C. Partial deprotection of the compound was observed after heating for about 4.5 hours.

### Example 10

### Preparation of Compound 21TB by TBDMS Deprotection with CsF

Compound 20TB (300 mg) was dissolved in acetonitrile (10 ml). CsF (70 mg) as added to the solution, forming a slurry. The slurry was heated at about 75° C. for about 17 hours, at which point a complete deprotection of the material was observed.

### Example 11

### Preparation of Compound 21TB by TBDMS Deprotection with Tetrabutylammonium Fluoride of 20TB

Compound 20TB (5 g) was dissolved in THF (40 mL) and tetrabutylammonium fluoride was added dropwise as 1M solution in THF (8.46 mL). The mixture was stirred for 1 hour at room temperature and the solvent was removed under reduced pressure. Toluene (300 ml) was added to the residue. The solution was washed with NaHCO₃ saturated solution (50 mL×3) and concentrated under reduced pressure resulting in crude 21 TB.

### Example 12

### Preparation of Compound 21TB in Methanesulfonic Acid/Methanol

A solution of methanesulphonic acid (15 mL, 0.2M in methanol/water, 10:1) was added to a solution of compound 20TB (3 g) in methanol (15 mL). The reaction mixture was stirred at 30° C. for about 3 hours, until monitoring by TLC (Hexane/EtAc, 4:1) indicated full consumption of the starting material.

Toluene (200 mL) was added to the reaction mixture and the reaction mixture was washed with a saturated NaHCO₃ solution (50 mL×2), forming an organic layer.

The organic layer was dried over MgSO₄ and the solvent was removed under reduced pressure to yield compound 21TB (2.97 g).

### Example 13

### Preparation of Compound 21TB by TBDMS Deprotection with Methanesulphonic Acid in Methanol

A solution of methanesulphonic acid (1.66 g) in methanol (200 ml) and water (19 ml) was added to a solution of 20TB (20.26 g, 81.2% assay) in methanol (185 ml). The resulting mixture was stirred at about 30° C. After 10.5 hours the HPLC indicated that the level of the starting material was 6% (on area), and the solution was cooled to room temperature.

EtOAc (400 mL) was added and the solution was washed with brine (400 mL). The organic layer was then washed with a saturated solution of NaHCO₃ (2×200 mL) and finally with brine (2×100 ml).

The organic layer was dried over Na₂SO₄ and the solvent was removed under reduced pressure to obtain 21TB (19.9 g).

### Example 14

### Preparation of Compound 21M by TBDMS Deprotection with Methanesulphonic Acid in Methanol

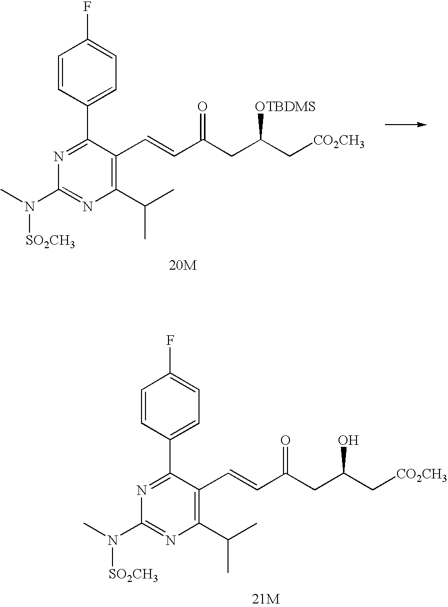

A solution of methanesulphonic acid (50 mL, 0.2 M in methanol/water, 10:1) was added to a solution of compound 20M (10 g) in methanol (50 mL), forming a reaction mixture. The reaction mixture was stirred at about 30° C. for about four hours. Methanesolfonic acid was added (0.35 ml) to the reaction mixture and the reaction mixture was stirred until completion of the reaction.

A product was extracted with toluene (2×100 mL) and washed with a saturated NaHCO₃ solution (100 mL), forming an organic layer. The organic layer was dried over MgSO₄ and the solvent was removed under reduced pressure, yielding 9.15 g of an oil.

### Example 15

### Extraction of Compound 21TB

A 1 L flask equipped with a mechanical stirrer was charged with crude 21 TB (41.6 g, assay=40.8%), toluene (200 mL), ethanol (200 mL), heptane (200 mL), and water (200 mL), forming a suspension. The suspension was stirred at room temperature until a clear solution was obtained. The solution was then poured into a separating funnel to allow phase separation. The EtOH/H₂O phase was removed. The toluene/heptane phase was then washed 4 times with a mixture of EtOH/H₂O (400 mL:200 mL), and the fractions were collected. Fractions 2-5 were combined and concentrated under reduced pressure to obtain an oily residue of purified 21TB(24.2 g, assay=56.0%, yield of 80%).

### Example 16

### Preparation of Compound 22TB (TBRE)

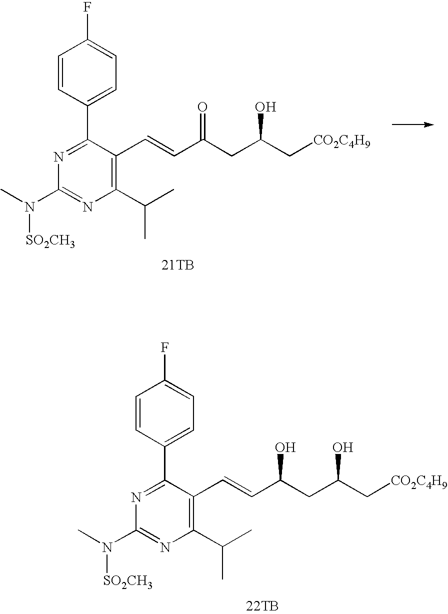

To a solution of 21TB (1 g) in dry THF (26 mL) and dry methanol (7 mL), a solution of diethylmethoxyborane (1M) in THF (2 mL) was added at about −78° C., forming a reaction mixture. The reaction mixture was stirred for 0.5 hour, NaBH₄ was added, and the stirring was continued for 3 hours. Acetic acid (1.2 mL) was added to the reaction mixture and the reaction mixture was warmed to ambient temperature.

Ethyl acetate (150 mL) was added to the reaction mixture and the pH was adjusted to 8 by addition of concentrated NaHCO₃ water solution. The layers were separated, and water was extracted by adding an additional amount of ethyl acetate (50 mL). The organic layers were combined and dried over MgSO₄. The solvents were then evaporated under reduced pressure, leaving a residue. The residue was treated with methanol and then the methanol was evaporated. Methanol treatment and evaporation was performed two more times, yielding crude compound 22TB (TBRE) (0.87 g, 86%).

### Example 17

### Conversion of Compound 22TB into Rosuvastatin Ca with Extraction in Ethyl Acetate

A 1 L reactor equipped with a mechanical stirrer was charged with EtOH (3 L), water (1800 mL), and TBRE (600 g), forming a reaction mixture. NaOH (47%, 1.2 eq, 114 g) was slowly added to the reaction mixture, at RT. The reaction mixture was stirred at about RT for two hours. The reaction mixture was filtered under reduced pressure with Synter and Hyflo to eliminate the small particles present. The reaction mixture was concentrated under reduced pressure at about 40° C. until half the volume of the reaction mixture remained.

Water (2000 mL) was added to the reaction mixture and the reaction mixture was stirred at about RT for 5 minutes. An aqueous phase and an organic phase formed. The phases were separated, and the aqueous phase was washed with ethyl acetate (3000 mL) and stirred at RT for half an hour. The organic phase was discarded.

The aqueous phase was concentrated under reduced pressure at about 40° C. until half the volume remained. Water (2800 mL) was added to the aqueous phase and the aqueous phase was stirred at about RT for 5 minutes. CaCl₂ (124 g) was added to the aqueous phase in portions over a period of about 10 minutes at a temperature of about RT. The aqueous phase was then stirred at about RT for about 1 hour, filtered, and washed with 1200 mL of water, yielding a powdery compound (491 g, 88%).

### Example 18

### Conversion of Compound 22TB into Rosuvastatin Ca with Extraction in Toluene

A 500 mL reactor equipped with a mechanical stirrer was charged with EtOH (150 mL), water (90 mL), and 22TB (30 g), forming a reaction mixture. NaOH (47%, 1.2 eq, 5.7 g) was slowly added to the reaction mixture at a temperature of about RT. The reaction mixture was stirred at RT for about 2 hours. The reaction mixture was filtered under reduced pressure with Synter and Hyflo to eliminate the small particles present. The reaction mixture was washed with toluene (150 mL) and stirred at RT for about half an hour, forming an aqueous phase and an organic phase. The two phases were separated, and the organic phase was discarded.

The aqueous phase was concentrated under reduced pressure at about 40° C. until half the volume remained. Water (104 mL) was added to the aqueous phase and the aqueous phase was stirred at about RT for 5 minutes. CaCl₂ (6.2 g) was added dropwise to the aqueous phase over 1 minute at about RT. The aqueous phase was then stirred at RT for about 1 hour, filtered, and washed with 1200 mL of water, yielding a powdery compound (26 g, 92%).

### Example 19

### Conversion of Compound 22TB (TBRE) into Rosuvastatin Ca with Extraction in Toluene

A 1 L reactor equipped with a mechanical stirrer was charged with EtOH (300 mL), water (90 ml), and 22TB (60 g), forming a reaction mixture. NaOH (47% 1.2 eq, 11.4 g) was added dropwise to the reaction mixture at RT. The reaction mixture was stirred at about RT for two hours. The reaction mixture was filtered under reduced pressure with Synter and Hyflo to eliminate the small particles present. Water (420 ml) was added to the reaction mixture.

The mixture was then extracted with toluene (3000 mL) and stirred at RT for half an hour. An aqueous phase formed and was isolated. The aqueous phase was concentrated under reduced pressure at 40° C. to half of the volume. Half of the remaining aqueous phase was transferred to a 500 mL reactor and water (110 mL) was added, creating a solution. The solution was stirred at RT for 5 minutes. Ca(OAc)₂ (8.8 g) was added dropwise to the solution over 1 min. at RT. The solution was stirred at RT for 1 hour, filtered, and washed with 60 mL of water, yielding a powdery compound (26 g, 94%).

What is claimed is:

1. A process for preparing compound 17, of the following structure: 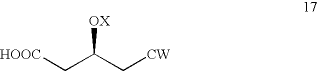
wherein W is a carboxyl protecting group and X is a hydroxyl protecting group, comprising: providing a solution of compound I of the following structure: 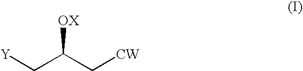
wherein Y is a C₁-C₄ ester, W is a carboxyl protecting group, and X is a hydroxyl protecting group, and a polar solvent; combining the solution with a base to obtain a reaction mixture with a pH of about 10 to about 13; and recovering compound 17.

2. The process of claim 1, wherein the polar solvent is selected from the group consisting of C₁₋₄ alcohols, nitrites, acetone, dioxane, and THF.

3. The process of claim 2, wherein the polar solvent is methanol or ethanol.

4. The process of claim 1, wherein the polar solvent is present in an amount of about 2 to about 15 volumes relative to compound I.

5. The process of claim 4 wherein the polar solvent is present in an amount of about 5 to about 10 volumes relative to compound I.

6. The process of claim 5, wherein the polar solvent is present in an amount of about 5 volumes relative to compound I.

7. The process of claim 1, wherein the base is selected from the group consisting of mono-, di-, tri-(C₁₋₄ alkyl)amino pyridines, mono-, di-, tri-(C₁₋₄ alkyl)amines, alkali metals, alkali earth hydroxides, alkali earth alkooxides, and C₁₋₄ alkyl lithium carbonates.

8. The process of claim 7, wherein the base is at least one of sodium hydroxide, potassium hydroxide or lithium hydroxide.

9. The process of claim 8, wherein the base is sodium hydroxide.

10. The process of claim 1, wherein the base is present in a concentration of about 0.9 to about 1.8 volumes relative to compound I.

11. The process of claim 10, wherein the base is present in a concentration of about 1.2 volumes relative to compound I.

12. The process of claim 1, wherein the base is added drop-wise.

13. The process of claim 1, wherein the reaction mixture obtained after combining the solution [symbol] the base is heated at a temperature of about 30° C. to about 70° C.

14. The process of claim 13, wherein the reaction mixture is heated at about 45° C. to about 55° C.

15. The process of claim 1, wherein the step of recovering compound 17 comprises: providing a solution of crude compound 17; partially evaporating the solvent; adding water; washing with a C₅-C₇ alkyl; extracting using an organic solvent selected from the group of: saturated or aromatic C₅-C₁₂ hydrocarbons, mono-, di-, tri-(C1 to C4)alkyl substituted benzene; acidifying the mixture using an inorganic acid to a pH of about 7 to about 5; and recovering compound 17.

16. A process for preparing rosuvastatin and salts thereof comprising preparing compound 17 according to the process of claim 1, and converting it to rosuvastatin or salts thereof.

17. A process of preparing compound 18, having the following structure: 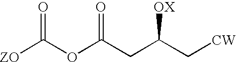
wherein W is a carboxyl protecting group, X is a hydroxyl protecting group and Z is a C₁₋₈ alkyl, comprising: adding a first solution comprising compound 17 of the following structure 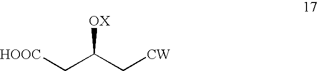
wherein W is a carboxyl protecting group and X is a hydroxyl protecting group, a first organic solvent and a base, to a second solution comprising a mono-, di-, tri-(C1 to C4)alkyl substituted benzene chloroformate, saturated or aromatic C₅-C₁₂ chloroformate or C₁₋₈ alkyl chloroformate and a second organic solvent to obtain a reaction mixture at a temperature of about −50° C. to about −10° C.; and maintaining the reaction mixture for a sufficient period of time to obtain compound 18.

18. The process of claim 17, wherein the base is an organic base.

19. The process of claim 18, wherein the base is selected from the group consisting of di(C₁ to C₄ alkyl) pyridine, mono-, di-, or tri-(C₁ to C₄ alkyl)amines, alkaline earth metals, alkaline earth hydroxides, alkaline earth alkoxides and C₁-C₄ alkyl lithium.

20. The process of claim 19, wherein the base is triethylamine.

21. The process of claim 17, wherein the first and second organic solvents are selected from the group consisting of saturated or aromatic C₅₋₁₂ hydrocarbons, mono-, di-, tri-,(C₁₋₄)alkyl substituted benzenes, and benzenes.

22. The process of claim 21, wherein the first and second organic solvents are selected from the group consisting of THF, toluene, methylene chloride, diethylether, benzene, and chloroform.

23. The process of claim 22, wherein the first and second organic solvents are toluene or THF.

24. The process of claim 17, wherein the first and second organic solvents are the same.

25. The process of claim 17, wherein the C₁₋₈ alkyl chloroformate is a C₁₋₄ alkyl chloroformate.

26. The process of claim 25 wherein the C₁₋₄ alkyl chloroformate is ethyl chloroformate or methyl chloroformate.

27. The process of claim 26, wherein the C₁₋₄ alkyl chloroformate is ethyl chloroformate.

28. The process of claim 17, wherein the molar ratio of the chloroformate to compound 17 in the reaction mixture is about 1 mole to about 3 moles.

29. The process of claim 28, wherein the molar ratio of the chloroformate to compound 17 in the reaction mixture is about 1 mol to about 1.5 mol.

30. The process of claim 17, wherein the first solution is combined with the second solution at a temperature of about −50° C. to about −30° C.

31. The process of claim 30, wherein the temperature is about −45° C. to about −40° C.

32. The process of claim 17, wherein the reaction mixture is maintained with gradual heating to about −10° C. to about 30° C.

33. The process of claim 30, wherein the reaction mixture is maintained with gradual heating to about 0° C.

34. The process of claim 17, wherein compound 18 of the following structure 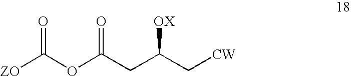
wherein W is a carboxyl protecting group, X is a hydroxyl protecting group and Z is a C₁₋₈ alkyl, is recovered from the reaction mixture.

35. A process for preparing rosuvastatin and salts thereof comprising preparing compound 18 according to the process of claim 17, and converting it to rosuvastatin or salts thereof.

36. A process for preparing compound 20 of the following structure 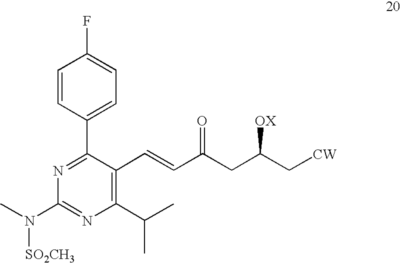
wherein W is a carboxyl protecting group and X is a hydroxyl protecting group, comprising providing compound 19 of the following structure 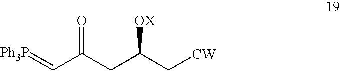
wherein W is a carboxyl protecting group and X is a hydroxyl protecting group, compound 14 of the following structure 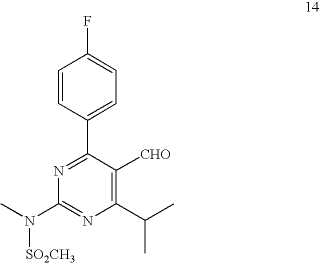
and a suitable organic solvent other than acetonitrile, to obtain a reaction mixture in an inert atmosphere; and heating the reaction mixture at about 70° C. to about reflux to obtain compound 20.

37. The process of claim 36, wherein the organic solvent is selected from the group consisting of saturated and aromatic C₅-C₁₂ hydrocarbons, mono-, di-, tri-(C₁ to C₄)alkyl substituted benzenes, and benzenes.

38. The process of claim 36, wherein compound 19 is present in an amount of 1.5 equivalents relative to compound 14.

39. The process of claim 36, wherein the organic solvent is toluene.

40. The process of claim 36, wherein the organic solvent is present in an amount of about 10 volumes relative to compound 14.

41. The process of claim 36, wherein the reaction mixture is heated at about 70° C. to about 110° C.

42. The process of claim 41, wherein the reaction mixture is heated at about 70° C. to about 110° C.

43. The process of claim 36, wherein compound 14 is present in compound 20 in a quantity of less than 5% as measured by HPLC.

44. The process of claim 42, wherein compound 14 is present in compound 20 in a quantity of less than 2% as measured by HPLC.

45. The process of claim 36, wherein triphenylphosphine oxide is formed and removed from the reaction mixture.

46. The process of claim 36, wherein compound 20 is further converted into compound 21, of the following structure: 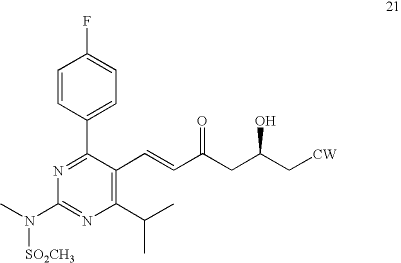
wherein W is a carboxyl protecting group.

47. A process of recovering compound 21 of the following structure 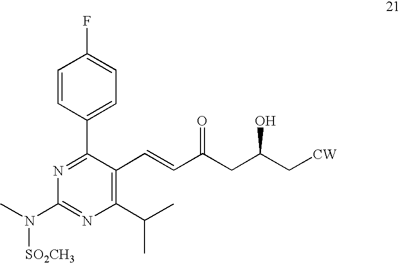
wherein W is a carboxyl protecting group, comprising: providing a two-phased system comprised of a mixture of a non-polar aliphatic solvent and a non-polar aromatic solvent and a mixture of a mixture of a lower aliphatic alcohol and water, each in an amount of about 4 to about 6 volumes relative to compound 21 and crude compound 21; washing the non-polar phase with a mixture of lower aliphatic alcohol and water; and recovering compound 21 from the organic phase.

48. The process of claim 47, wherein the compound 21 recovered has a purity of greater than about 80% as determined by HPLC.

49. The process of claim 48, wherein the compound 21 recovered has a purity of greater than about 90% as determined by HPLC.

50. The process of claim 49, wherein the yield is greater than about 90%.

51. The process of claim 50, wherein the yield is greater than about 95%.

52. The process of claim 47, wherein the non-polar aliphatic solvent, non-polar aromatic solvent, lower aliphatic alcohol and water are each present in an equal volume of about 5 volumes relative to compound 21.

53. The process of claim 47, wherein the non-polar aliphatic solvent is heptane.

54. The process of claim 47, wherein the non-polar aromatic solvent is toluene.

55. The process of claim 47, wherein the lower aliphatic alcohol is ethanol.

56. The process of claim 47, wherein the two-phase system is obtained by mixing at room temperature until a clear solvent is obtained at which point the mixture is allowed to separate into phases.

57. The process of claim 47, wherein washing the non-polar phase with the mixture of polar solvent and water is in a plurality of portions.

58. The process of claim 57, wherein washing is in about 4 to about 5 portions.

59. The process of claim 47, wherein the ratio of ethanol to water is about 2:1 by volume.

60. The process of claim 47, wherein the ethanol is present in an amount of about 4 to about 6 volumes relative to compound 21.

61. The process of claim 60, wherein the ethanol is present in an amount of about 5 volumes relative to compound 21.

62. The process of claim 47, wherein the water is present in an amount of about 8 to about 12 volumes relative to compound 21.

63. The process of claim 62, wherein the water is present in an amount of about 10 volumes relative to compound 21.

64. A process for preparing rosuvastatin, and pharmaceutically acceptable salts thereof, comprising:
a. providing a solution of compound I of the following structure 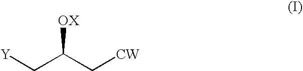
wherein Y is a C₁-C₄ ester, W is a carboxyl protecting group and X is a hydroxyl protecting group, and a polar solvent;
b. combining the solution with a base to obtain a pH of about 10 to about 13 to form a first solution comprising compound 17 of the following structure 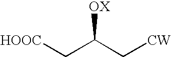
wherein W is a carboxyl protecting group and X is a hydroxyl protecting group;
c. adding a second solution comprising a mono-, di-, tri-(C₁ to C₄)alkyl substituted benzene chloroformate, saturated or aromatic C₅-C₁₂ chloroformate or C₁₋₈ alkyl chloroformate and an organic solvent to obtain a reaction mixture while maintaining a temperature of about −50° C. to about −10° C.;
d. maintaining the reaction mixture for a sufficient period of time to obtain compound 18 of the following structure 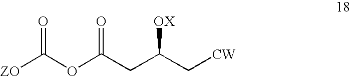
wherein W is a carboxyl protecting group, X is a hydroxyl protecting group and Z is a C₁₋₈ alkyl;
e. converting compound 18 into compound 19 of the following structure 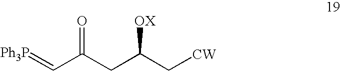
wherein W is a carboxyl protecting group and X is a hydroxyl protecting group;
f. combining compound 19 and compound 14 of the following structure 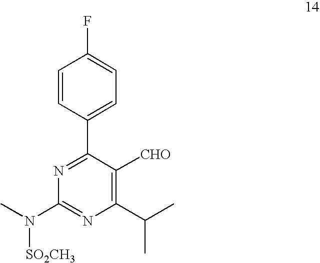
and a suitable organic solvent other than acetonitrile, to obtain a reaction mixture in an inert atmosphere such as argon or nitrogen;
g. heating the reaction mixture at about 70° C. to about reflux for period to obtain compound 20 of the following structure 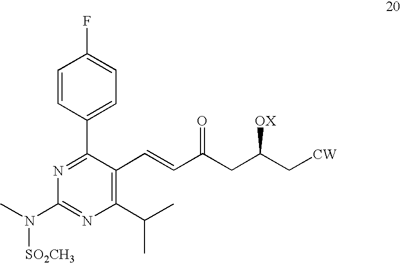
wherein W is a carboxyl protecting group and X is a hydroxyl protecting group;
h. converting compound 20 into compound 21 of the following structure 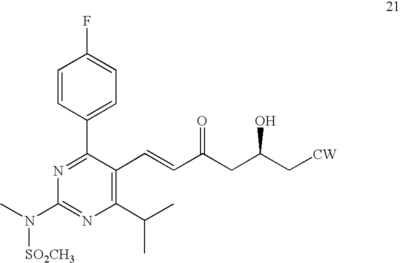
wherein W is a carboxyl protecting group;
i. optionally recovering compound 21 by providing a two-phased system comprised of a mixture of a non-polar aliphatic solvent and a non-polar aromatic solvent and a mixture of a mixture of a lower aliphatic alcohol and water, each in an amount of about 4 to about 6 volumes relative to compound 21 and crude compound 21, washing the non-polar phase with a mixture of lower aliphatic alcohol and water, and recovering compound 21 from the organic phase;
j. converting compound 21 into compound 22 of the following structure 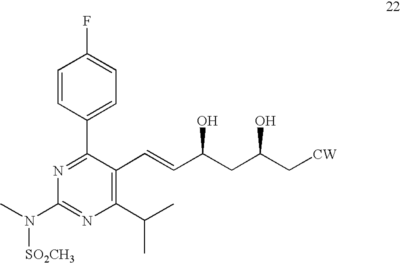
wherein W is a carboxyl protecting group; and
k. converting compound 22 into rosuvastatin.

65. The process of claim 64, wherein:
a. compound 17 of the following structure 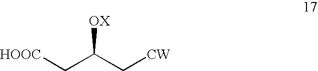
wherein W is a carboxyl protecting group and X is a hydroxyl protecting group, is recovered from step b. by partially evaporating the solvent from the first solution; adding water; washing with a C₅₋₇ alkyl; extracting using an organic solvent selected from the group consisting of: saturated or aromatic C₅-C₁₂ hydrocarbons, mono-, di-, tri-(C₁ to C₄)alkyl substituted benzenes, acidifying the mixture using an inorganic acid to a pH of about 7 to about 5; and recovering compound 17 from the organic phase; and
b. compound 17 is combined with a first organic solvent and a base to form the first solution comprising compound 17.

66. The process of claim 64, wherein the rosuvastatin obtained is further converted to a pharmaceutically acceptable salt of rosuvastatin.

67. The process of claim 66, wherein the salt of rosuvastatin is the calcium salt.
